(19) United States
(12) Patent Application Publication
(10) Pub. No.: US 2009/0204048 A1
(43) Pub. Date: Aug. 13, 2009

(54) Easy removable bandage

(76) Inventor: Samuel Spector, Edison, NJ (US)

Correspondence Address: SAMUEL SPECTOR, 14 BROOKFALL ROAD, EDISON, NJ, 08817-2947, US

(21) Appl. No.: 12/313,059

(22) Filed: Nov. 18, 2008

Related U.S. Application Data
(60) Provisional application No. 61/065,023, filed on Feb. 8, 2008.

(51) Int. Cl. A61F 13/02

(52) U.S. Cl. 602/57

(57) ABSTRACT

Self adhering type of a bandage having a tab or tabs without adhesive coatings, to facilitate easy removal from a wound. As an alternate for easy removal, self adhering bandage having areas at the inner surface edges without adhesive coatings and distinguishing markings and/or colors on the opposite outer uncoated side to locate the uncoated areas.

### BACKGROUND OF THE INVENTION

The invention involves an improvement to self adhering type bandages as noted in my Provisional Patent Application No. 61/065,023. These types of bandages are generally composed of an elastic plastic, fabric, cloth or other suitable material having a pressure sensitive adhesive coating and an absorbent pad on one side, to cover a wound. The problem with these type bandages is that they are sometimes difficult to remove, especially if one is wearing medical gloves.

It is therefore the object of the invention to provide a bandage which can be easily removed from the skin.

### SUMMARY OF THE INVENTION

The invention includes a bandage having one or more projecting tabs. The tabs do not have any adhesive coatings and may have distinguishing markings and/or colors for locating the tabs. An alternate is leaving a small area of one or more ends of a bandage without an adhesive coating and having distinguishing markings and /or colors on the outer uncoated surface of the bandage to locate the area of the bandage without the adhesive. Said bandage with tabs or the alternate bandage with no adhesive coatings at the ends, allow for easy removal of the bandages from the skin.

### BRIEF DESCRIPTION OF THE DRAWINGS

FIG. 1 shows the adhesive coating and pad on the inner surface of a bandage with projecting tabs, the tabs being uncoated in accordance with the invention, and which are an extension of the flexible bandage material.

FIG. 2 shows the outer uncoated surface of the bandage in FIG. 1, and includes distinguishing markings and/or colors of the tabs.

FIG. 3 shows the adhesive coating and pad on the inner surface of a bandage with projecting tabs, the tabs being fastened to the bandage on the outer surface as shown in FIG. 4.

FIG. 4 shows the outer uncoated surface of the bandage in FIG. 3, and the tabs mounted on the outer uncoated side

FIG. 5 shows the adhesive coating and pad on the inner surface of a bandage with the ends of the bandage without the adhesive coating.

FIG. 6 shows the outer uncoated surface of the bandage in FIG. 5, and includes distinguishing markings and/or colors which locate the uncoated area shown in FIG. 5.

### DETAILED DESCRIPTION OF THE DRAWINGS

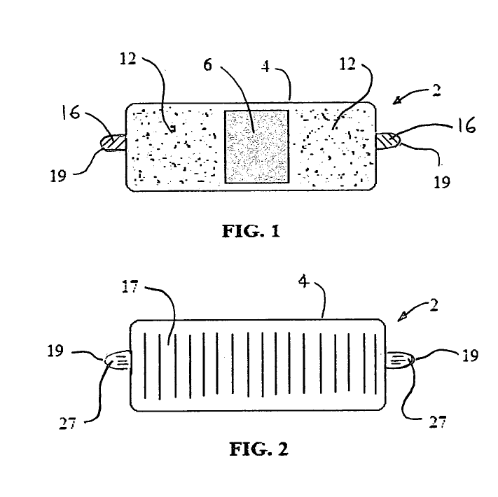

FIG. 1 shows a bandage 2 having a flexible strip 4 with an inner surface having pressure sensitive adhesive coatings 12 and a pad 6. At the ends of the strip are tabs 19 with uncoated areas 16. FIG. 2 shows the outer uncoated surface 17 of the bandage 2. The tabs 19 are shown with distinguishing markings and/or colors 27. It may be determined from actual experience that in some cases the tabs may not require any special markings and/or colors.

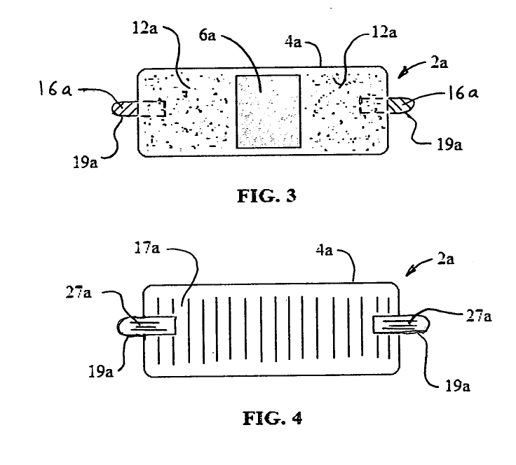

FIG. 3 shows a bandage 2a having a flexible strip 4a with an inner surface having pressure sensitive adhesive coatings 12a and a pad 6a. Projecting from the strip are tabs 19a with uncoated areas 16a. In FIG. 4 the tabs 19a are fastened to the outer uncoated surface 17a of the bandage 2a. The tabs 19a have distinguishing markings and/ or colors 27a.

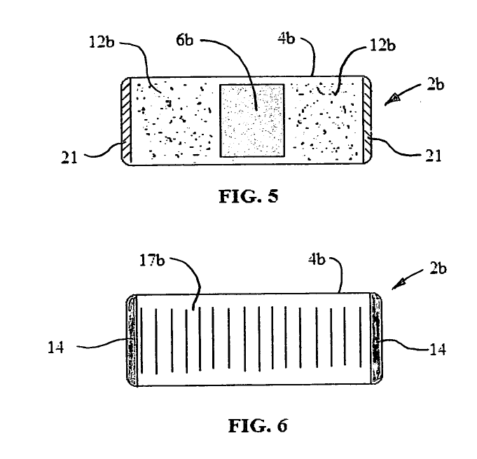

FIG. 5 shows a bandage 2b having a flexible strip 4b with an inner surface having pressure sensitive adhesive coatings 12b and a pad 6b. The ends of the strip 21 are uncoated. FIG. 6 shows the outer uncoated surface 17b of the bandage 2b and includes distinguishing markings and/or colors 14 which are opposite and locate the inner uncoated surface area 21 shown in FIG. 5.

Although the drawings show one type of bandage, the invention is applicable to any self adhering type bandage. Said bandages being made in many sizes and configurations.

I claim:

1. A bandage comprised of a flexible material having an inner and outer surface, a pad secured to the inner surface and adhesive coatings on the same side as the pad, and the outer surface being without any adhesive coatings; the edge of the flexible material having one or more projecting tabs.

2. The tabs indicated in claim 1 are an extension of the flexible material.

3. The tabs indicated in claim 1 are a separate material fastened to the inner or outer surface of the flexible material.

4. The tabs noted in claim 2 & 3 have no adhesive coatings on either surface and have distinguishing markings and/or colors on the outer surface.

5. The tabs noted in claim 2 & 3 and have no adhesive coatings on either surface, and on the outer surface are the same color and appearance as the flexible material.

6. A bandage comprised of a flexible material having an inner and outer surface, a pad secured to the inner surface and adhesive coatings on the same side as the pad; one or more areas on the inner surface, extending to the edge of the flexible material, are without adhesive coatings.

7. The outer surface of the uncoated areas, as noted in claim 6, have distinguishing markings and/or colors for locating the uncoated areas on the inner surface of the bandage.
